(19) United States
(12) Patent Application Publication
(10) Pub. No.: US 2008/0249426 A1
(43) Pub. Date: Oct. 9, 2008

(54) Palm type electrocardiograph

(75) Inventors: Bo-Jau Kuo, Taipei (TW); Chin Teng Lin, Hsinchu City (TW); Hsin Yen, Clifton, VA (US); Khein Seng Pua, Jhudong Township (TW)

Correspondence Address: BACON & THOMAS, PLLC, 625 SLATERS LANE, FOURTH FLOOR, ALEXANDRIA, VA, 22314-1176, US

(73) Assignee: GTMRI, Inc., Clifton, VA (US)

(21) Appl. No.: 11/730,954

(22) Filed: Apr. 5, 2007

(51) Int. Cl. A61B 5/0404

(52) U.S. Cl. 600/523

(57) ABSTRACT

The present invention provides a palm type electrocardiograph having a case, in which a push button is provided at the front side thereof, a USB port is installed along a lateral side thereof, and two metal holes are assembled at the back thereof. A conductive sticker is also designed with two metal electrodes being penetrately assembled in the center thereon. The conductive sticker has three layers, the first layer is foam, the second layer is conductive graphite, and the third layer is conductive adhesive. An internal circuit is provided in the palm type electrocardiograph, including a microprocessor, an A/D converter, a program memory and a flash memory.

### FIELD OF THE INVENTION

The present invention relates to a design of a palm type electrocardiograph, and more particularly to a palm type electrocardiograph for catching electrocardiogram by two electrodes.

### BACKGROUND OF THE INVENTION

Electrocardiogram is important information for medical treatment that shows personal heart rate for evaluating the personal heart function and the personal response to a medicine

Electrocardiogram is generally obtained via electrocardiograph in the hospital by a doctor. It is impossible that each patient owns an electrocardiograph, since the electrocardiograph used in the hospital is too big to carry personally. Three electrodes are generally used by electrocardiograph to catch electrocardiogram, the wires between the electrodes and the electrocardiograph are very long so as to generate noise easily.

As computer and Internet popularize as well as integrated circuit is widely used, the era for minimizing electrocardiograph begins. The objective of the present invention is to minimize electrocardiograph to a palm type.

### SUMMARY OF THE INVENTION

The present invention provides a palm type electrocardiograph having a case, in which a push button is provided at the front side thereof, a USB port is installed along a lateral side thereof, and two metal holes are assembled at the back thereof.

A conductive sticker is also designed with two metal electrodes being penetrately assembled in the center thereon. The conductive sticker has three layers, the first layer is foam, the second layer is conductive graphite, and the third layer is conductive adhesive. The conductive graphite and the conductive adhesive are each formed into two pieces, and the two metal electrodes are each penetrately assembled respectively through the two pieces so that the two metal electrodes do not short.

An internal circuit is provided in the palm type electrocardiograph, including a microprocessor, an AID converter, a program memory and a flash memory.

The operation steps of the palm type electrocardiograph are as follows: first to push the push button for enabling power; a program begins accepting electrocardio signals; the electrocardio signals are processed in the microprocessor and stored in the flash memory; if a push button signal is received again, it means that the user stops the operation, so the program terminates; if no push button signal is received, then calculate if electrocardio signals are recorded for five minutes, if five minutes are reached, stop the operation, otherwise continue recording.

### BRIEF DESCRIPTION OF THE DRAWINGS

FIG. 1 is a schematic view of the palm type electrocardiograph in accordance with the present invention;

FIG. 2 is a schematic view of the conductive sticker in accordance with the present invention;

FIG. 3 is a block diagram of the internal circuit in the palm type electrocardiograph in accordance with the present invention.

FIG. 4 shows the operation flow chart of the palm type electrocardiograph in accordance with the present invention.

### DETAILED DESCRIPTION OF THE PREFERRED EMBODIMENT

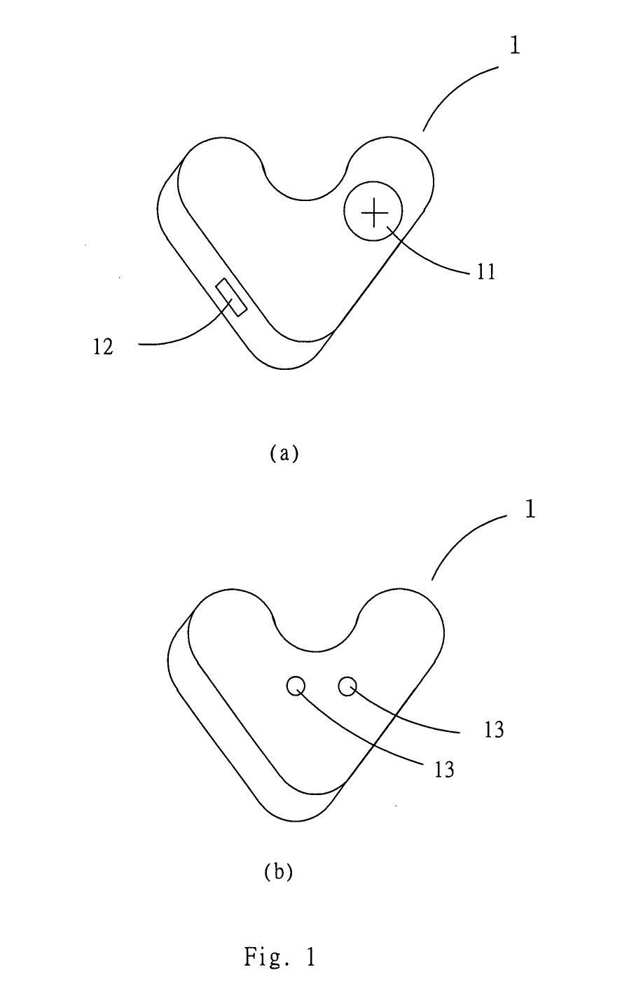

First referring to FIG. 1, which is a schematic view of the palm type electrocardiograph 1 in accordance with the present invention. FIG. 1(a) shows a front view of the palm type electrocardiograph 1 having a case, in which a push button 11 is provided at the front side thereof, a USB port 12 is installed along a lateral side thereof. FIG. 1(b) shows a back view of the palm type electrocardiograph having a case, in which two metal holes 13 are assembled at the center thereof.

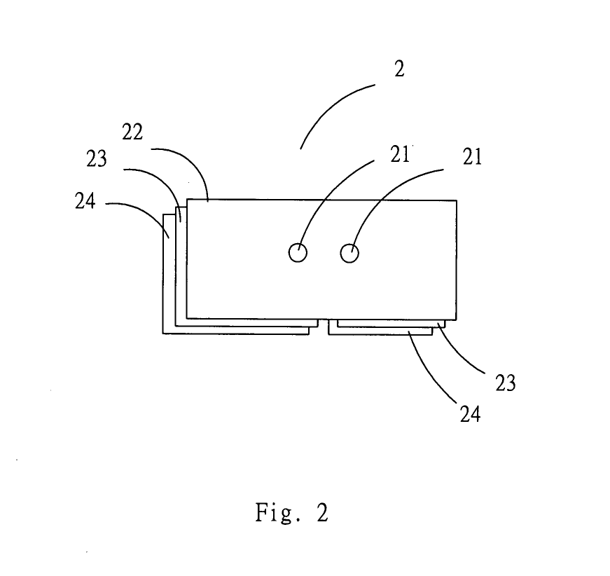

FIG. 2 is a schematic view of the conductive sticker 2 in accordance with the present invention. The conductive sticker 2 is designed with two metal electrodes 21 being penetrately assembled in the center thereon. The conductive sticker 2 has three layers, the first layer is foam 22, the second layer is conductive graphite 23, and the third layer is conductive adhesive 24. The conductive graphite 23 and the conductive adhesive 24 are each formed into two pieces, and the two metal electrodes 21 are each penetrately assembled respectively through the two pieces so that the two metal electrodes 21 do not short. The conductive adhesive 24 contains AgCl.

A user plugs the two metal electrodes 21 of the conductive sticker 2 into the two metal holes 13 respectively to form a so-called biosensor 30, and the palm type electrocardiograph 1 is therefore combined with the conductive sticker 2, then the user attaches the conductive sticker 2 around the heart thereof, push the push button 11 for recording the electrocardio signals. After the recording is finished, a USB wire is used for connecting the USB port 12 and a computer to display the electrocardiogram. (The computer must have software to display the electrocardiogram.)

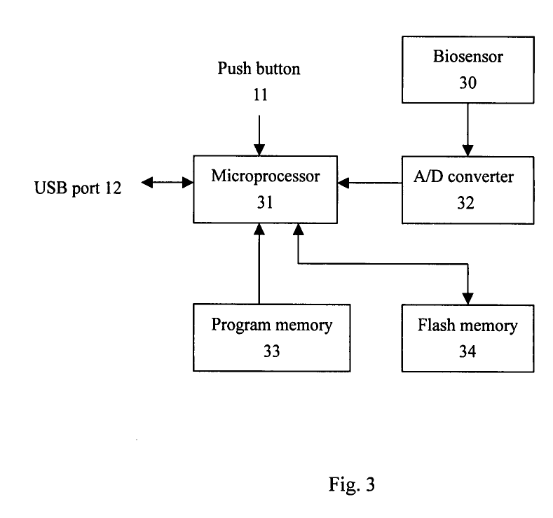

An internal circuit is provided in the palm type electrocardiograph 1, as shown in FIG. 3, including a microprocessor 31, an A/D converter 32, a program memory 33 and a flash memory 34. The electrocardio signals sensed by the biosensor 30 are inputted to the A/D converter 32 for converting into digital signals, and then inputted to the microprocessor 31 for processing. The microprocessor 31 accepts the program control of the program memory 33 for processing the digital signals outputted from the A/D converter 32, and stores the result to the flash memory 34. Thereafter a USB wire is used for connecting the USB port 12 and a computer to display the electrocardiogram stored in the flash memory 34. (The computer must have software to display the electrocardiogram.)

A microprocessor PS 2101 of Phison Electronics Corp. is used to act as the microprocessor 31 of the palm type electrocardiograph 1. AD8607ARMZ (A/D converter) and AD7468BRTZ (amplifier) of ADI Company are used to function as the A/D converter 32. EN29LV512 (EEPROM) of Eon Silicon Solution Inc. is used to act as the program memory 33. TH58NVG2S3BTG00 of TOSHIBA is used to function as the flash memory 34. A rechargeable battery UP042035-PK1 of Usance Technology Co., Ltd is used as the direct current power supply.

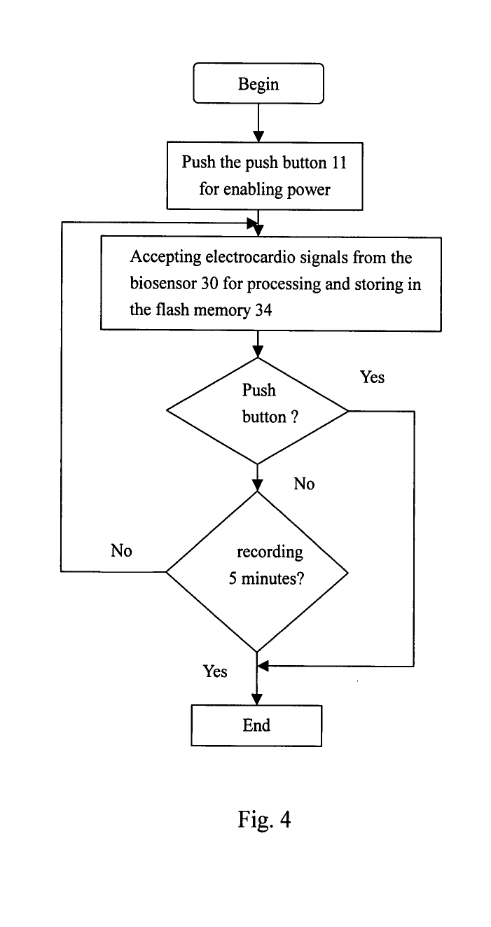

The operation flow chart of the palm type electrocardiograph 1 is shown in FIG. 4. First to push the push button 11 for enabling power; a program begins accepting electrocardio signals via the biosensor 30; the electrocardio signals is processed in the miroprocessor 31 and stored in the flash memory 34; if a push button signal is received again, it means that the user stops the operation, so the program terminates; if no push button signal is received, then calculate if electrocardio signals are recorded for five minutes, if five minutes are reached, stop the operation, otherwise continue recording.

The scope of the present invention depends only upon the following claims, and are not limited by the above embodiment.

What is claimed is:

1. A palm type electrocardiograph, comprising:
a case, wherein a push button is provided at the front side thereof, a USB port is installed along a lateral side thereof, and two metal holes are assembled at the back thereof;
a conductive sticker, wherein two metal electrodes being penetrately assembled in the center thereon;
an internal circuit, including a microprocessor, an A/D converter, a program memory and a flash memory.

2. The palm type electrocardiograph according to claim 1, wherein the conductive sticker has three layers, a first layer is foam, a second layer is conductive graphite, and a third layer is conductive adhesive; the conductive graphite and the conductive adhesive are each formed into two pieces, and the two metal electrodes are each penetrately assembled respectively through the two pieces so that the two metal electrodes do not short; the conductive adhesive contains AgCl.

3. The palm type electrocardiograph according to claim 1, wherein after the two metal electrodes are plugged into the two metal holes respectively and the conductive sticker is attached around a human heart, push the push button so that the internal circuit accepts electrocardio signals via the two metal electrodes for processing and storing in the flash memory; thereafter a USB wire can be used for connecting the USB port and a computer to display the electrocardio signals stored in the flash memory.

4. The palm type electrocardiograph according to claim 1, wherein a microprocessor PS 2101 of Phison Electronics Corp. is used to act as said microprocessor of the palm type electrocardiograph; AD8607ARMZ (A/D converter) and AD7468BRTZ (amplifier) of ADI Company are used to function as the A/D converter; EN29LV512 (EEPROM) of Eon Silicon Solution Inc. is used to act as the program memory; TH58NVG2S3BTG00 of TOSHIBA is used to function as the flash memory; a rechargeable battery UP042035-PK1 of Usance Technology Co., Ltd is used as a direct current power supply.

5. The palm type electrocardiograph according to claim 3, wherein a operation steps of the palm type electrocardiograph is as follows: first to push the push button for enabling power; a program begins accepting electrocardio signals via the two metal electrodes; the electrocardio signals is processed in the miroprocessor and stored in the flash memory; if a push button signal is received again, it means that a user stops the operation, so the program terminates; if no push button signal is received, then calculate if electrocardio signals are recorded for five minutes, if five minutes are reached, stop the operation, otherwise continue recording.
